(19) United States
(12) Patent Application Publication
(10) Pub. No.: US 2025/0041335 A1
(43) Pub. Date: Feb. 6, 2025

(54) PERCARBONATE COMPOSITIONS AND USES THEREOF

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 18/791,306

(22) Filed: Jul. 31, 2024

Related U.S. Application Data
(60) Provisional application No. 63/529,981, filed on Jul. 31, 2023.

(51) Int. Cl. A61K 33/40; A23L 33/16; A61P 43/00

(52) U.S. Cl. CPC A61K 33/40; A23L 33/16; A61P 43/00

(57) ABSTRACT

Percarbonate and percarbonate compositions, for example a dietary supplement or a pharmaceutical composition, are disclosed along with their uses, such as improving physical performance of a subject, increasing recovery from physical exertion of a subject, reducing lactic acid burn of a subject, and/or preventing, treating, or curing a percarbonate-improvable condition of a subject that causes fatigue and/or malaise.

### RELATED APPLICATIONS

This application claims priority to and the benefit of U.S. provisional patent application 63/529,981, filed Jul. 31, 2023, titled “PERCARBONATE COMPOSITIONS AND USES THEREOF,” the entirety of the disclosure of which is hereby incorporated by this reference.

### FIELD OF THE INVENTION

The disclosure relates to percarbonate and specifically to oral percarbonate compositions and their uses for improving physical performance of a subject, increasing recovery from physical exertion of a subject, reducing lactic acid burn of a subject, and/or preventing, treating, or curing a percarbonate-improvable condition of a subject that causes decreased physical performance, fatigue and/or malaise.

### BACKGROUND OF THE INVENTION

Sodium percarbonate, or sodium carbonate peroxide (not to be confused with sodium bicarbonate or sodium carbonate) is a chemical substance with formula 2Na2CO3·3H2O2.

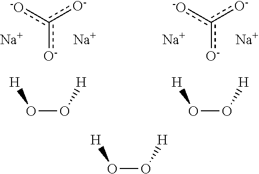

Sodium percarbonate is a colorless, crystalline, hygroscopic and water-soluble solid. It is sometimes abbreviated as SPC. It contains 32.5% by weight of hydrogen peroxide.

Sodium percarbonate is used in some eco-friendly bleaches and other cleaning products such as the Oxyclean® product. No use of it to produce any beneficial biological effects upon ingestion had ever been described, as the compound is considered a poison that should not be ingested. For example, products containing sodium percarbonate often will contain a poisoning warning containing the following language: “The ingestion of sodium percarbonate may cause bloating, belching, irritation of the mouth and throat, nausea, vomiting, abdominal pain and diarrhea”. In the United States, the Occupational Safety and Health Administration (OSHA) requires a “harmful if swallowed” label on containers of hazardous chemicals, so this label accompanies all sodium percarbonate products on the market.

### SUMMARY OF THE INVENTION

Percarbonate and percarbonate compositions for oral administration are disclosed.

The percarbonate may be sodium percarbonate. Surprisingly, it was found that administration of sodium percarbonate in doses of 2-10 grams in humans, preferably in capsule form, did not result in nausea, diarrhea or any other detrimental gastrointestinal effects. In fact, the subjects after ingesting sodium percarbonate before exercise experienced increased endurance, increased strength, decreased lactic acid “burn” (the burning sensation muscles get after anaerobic energy production has started in the body due to strenuous exercise) and improved recovery from exercise. It is also notable that this increase in energy did not have any appreciable stimulatory effects like those of caffeine and other stimulants which cause a false feeling of “energy” making it especially preferable for people that want to avoid caffeine and other CNS stimulants.

Compositions may further include a dietary ingredient, a food additive, or a pharmaceutically acceptable additive. The pharmaceutically acceptable additive may be one or more of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and a carrier. Specifically, the pharmaceutically acceptable additive may be one or more of calcium phosphate, cellulose, stearic acid, croscarmellose cellulose, magnesium stearate, and silicon dioxide. The compositions may be in capsule form.

A dietary supplement including a therapeutically effective dose of percarbonate is also described. The percarbonate may be sodium percarbonate. In some aspects, the amount of percarbonate in a dose of the dietary supplement is about 1 mg to about 30,000 mg. The dietary supplement may be in the form of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, or combinations thereof.

In some implementations, the dietary supplement additionally comprises one or more of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and an acceptable carrier. In some aspects, the acceptable carrier comprises an excipient, a lubricant, a binder, a disintegrator, a diluent, an extender, a solvent, a suspending agent, a dissolution aid, an isotonic agent, a buffering agent, a soothing agent, an amphipathic lipid delivery system, or combinations thereof.

A pharmaceutical composition including a therapeutically effective dose of percarbonate is additionally described. The percarbonate may be sodium percarbonate. The pharmaceutical composition may provide a daily dosage of percarbonate of 1 to 30,000 mg of creatine in one or multiple unit dosage forms. In some aspects, the unit dosage form is a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, or combinations thereof.

In some aspects, the pharmaceutical composition additionally comprises one or more of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and an acceptable carrier. In some implementations, the acceptable carrier comprises an excipient, a lubricant, a binder, a disintegrator, a diluent, an extender, a solvent, a suspending agent, a dissolution aid, an isotonic agent, a buffering agent, a soothing agent, an amphipathic lipid delivery system, or combinations thereof.

Methods of using percarbonate are also described. The method may include administering a percarbonate to a subject wherein an effective amount of the percarbonate is administered to the subject. The percarbonate may be sodium percarbonate. In some aspects, the method comprises administering a percarbonate to a subject wherein a daily dosage of percarbonate of 1 to 30,000 mg in one or multiple doses is administered to the subject. In some aspects, the daily dosage is administered in one dose. In other aspects, the daily dosage is administered in two to four doses. The route of administration is selected from topical, transdermal, oral, rectal, ophthalmic, nasal, vaginal, or parenteral routes. In particular aspects, percarbonate is administered orally. In some aspects, percarbonate is administered as a dietary supplement or as a pharmaceutical composition.

In some implementations, doses of percarbonate are in the form of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, or combinations thereof. In some aspects, the dose of percarbonate additionally comprises one or more of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and an acceptable carrier. In some implementations, the acceptable carrier comprises an excipient, a lubricant, a binder, a disintegrator, a diluent, an extender, a solvent, a suspending agent, a dissolution aid, an isotonic agent, a buffering agent, a soothing agent, an amphipathic lipid delivery system, or combinations thereof.

In some implementations, percarbonate is administered to a subject to treat a percarbonate-improvable condition, for example, alcohol use, drug use, malnutrition, fatigue side effects from narcotics, antihistamines, chemotherapy, radiation therapy, pain medicines, heart medicines, and antidepressants, lack of sleep, lack of physical exercise, overexhaustion, adrenal insufficiency, amyotrophic lateral sclerosis (ALS), anemia, anxiety disorders, cancer, myalgic encephalomyelitis/chronic fatigue syndrome (ME/CFS), chronic infection or inflammation, chronic kidney disease, chronic obstructive pulmonary disease (COPD), emphysema, coronavirus disease 2019 (COVID-19), mood disorders, depression (major depressive disorder), diabetes, fibromyalgia, grief, heart disease, heart failure, hepatitis A, hepatitis B, hepatitis C, HIV/AIDS, hyperthyroidism (overactive thyroid), hypothyroidism (underactive thyroid), inflammatory bowel disease (IBD), liver disease, low vitamin D, lupus, mononucleosis, multiple sclerosis, obesity, Parkinson's disease, physical abuse, emotional abuse, polymyalgia rheumatica, pregnancy, rheumatoid arthritis, sleep apnea, stress, and traumatic brain injury.

### DETAILED DESCRIPTION OF THE INVENTION

Detailed aspects and applications of the disclosure are further described below, in any drawings/structures, and in the claims. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant art, that the present disclosure may be practiced without these specific details. It should be noted that there are many different and alternative configurations, devices, methods, and technologies to which the divulged disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

It will be understood that the term “percarbonate” refers to an ionic compound that comprises the carbonate (CO₃²⁻) and the peroxo (H—O—O—H) moieties bonded to a cation. Although the most commonly used form of percarbonate is sodium percarbonate, other percarbonates may be used for the purposes of this disclosure, which can be prepared by electrolysis of the corresponding carbonate salts for example. For example, potassium percarbonate can be prepared by electrolysis of potassium carbonate, as described by Manorahan et al. (“Electrolytic Preparation of Sodium and Potassium Percarbonate”, Transactions of the SAEST, Vol. 35 No. 2, April-June 2000), which is fully incorporated by reference herein. While electrolysis is described as a method to produce other percarbonates such as potassium percarbonate, the methods of production are not limited to electrolysis. Other possible methods of production may include, for example, ion exchange between sodium percarbonate and a salt of a strong acid and weak base, such as ammonium chloride in a suitable ion exchange solvent or matrix under the right conditions to produce ammonium percarbonate. As non-limiting examples, other percarbonates suitable for use according to this disclosure, may include: potassium percarbonate, magnesium percarbonate, calcium percarbonate, lithium percarbonate, arginine percarbonate, creatine percarbonate, caffeine percarbonate, creatine percarbonate, creatinine percarbonate, arginine percarbonate, lysine percarbonate, histidine percarbonate, methylamine percarbonate, ethylamine percarbonate, propylamine percarbonate, butylamine percarbonate, pentylamine percarbonate, hexylamine percarbonate, heptylamine percarbonate, diethylamine percarbonate, spermidine percarbonate, putrescine percarbonate, trimethylamine percarbonate, isobutylamine percarbonate, isoamylamine percarbonate, pyrrolidine percarbonate, cadaverine percarbonate, agmatine percarbonate, phenethylamine percarbonate, hordenine percarbonate, trigonelline percarbonate, carnitine percarbonate, choline percarbonate, glycocyamine percarbonate, guanidine percarbonate, methylguanidine percarbonate, and dimethylguanidine percarbonate.

It will be understood that the term “percarbonate hydrate” refers to possible hydrates of percarbonate that can be formed after exposure of percarbonate to atmospheric humidity or after reaction of percarbonate with water to form percarbonate hydrate crystals. The hydrates formed can be a monohydrate, a dihydrate, a quasi-hydrate and the like.

As used herein, the term “pharmaceutically acceptable” is used in its broadest sense and may describe the quality of meeting Food and Drug Administration (FDA) standards, United States Pharmacopeial Standards (USP), US Department of Agriculture (USDA) standards for food-grade materials, commonly accepted standards of the nutritional supplement industry, industry standards, or botanical standards. These standards may delineate acceptable ranges of aspects of ingredients of a pharmaceutical composition such as edibility, toxicity, pharmacological effect, or any other aspect of a chemical, composition, or preparation used in implementations of a pharmaceutical composition.

As used herein, “pharmaceutically acceptable additive” or “additive” are terms used in their broadest sense. Particular implementations of the compositions described in this document may also comprise an additive (e.g. one of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically acceptable additives from the disclosure in this document. In particular implementations, pharmaceutically acceptable additives may include, by non-limiting example, calcium phosphate, cellulose, stearic acid, croscarmellose cellulose, magnesium stearate, and silicon dioxide.

As used herein, “effective”, “pharmaceutically effective”, or “therapeutically effective” are terms or phrases used in their broadest sense, including, by non-limiting example, effective in a clinical trial or for a specific subject or patient. When used in a method claim, effective, pharmaceutically effective, or therapeutically effective will mean in a dose enough to achieve the claim's preamble or purpose.

The disclosure relates to percarbonate and percarbonate compositions comprising percarbonate useful as a dietary supplement or pharmaceutical composition. In some aspects, percarbonate is combined with a dietary ingredient, a food additive, or a pharmaceutically acceptable additive.

The described percarbonates and percarbonate compositions may be administered to a subject for improving physical performance of a subject, increasing recovery from physical exertion of a subject, reducing lactic acid burn of a subject, and/or preventing, treating, or curing a percarbonate-improvable condition of a subject that causes decreased physical performance, fatigue and/or malaise.

For simplicity, such conditions will be referenced as “percarbonate-improvable conditions”. Percarbonate-improvable conditions include, but are not limited to: alcohol use, drug use, malnutrition, fatigue side effects from narcotics, antihistamines, chemotherapy, radiation therapy, pain medicines, heart medicines, and antidepressants, lack of sleep, lack of physical exercise, overexhaustion, adrenal insufficiency, amyotrophic lateral sclerosis (ALS), anemia, anxiety disorders, cancer, myalgic encephalomyelitis/chronic fatigue syndrome (ME/CFS), chronic infection or inflammation, chronic kidney disease, chronic obstructive pulmonary disease (COPD), emphysema, coronavirus disease 2019 (COVID-19), mood disorders, depression (major depressive disorder), diabetes, fibromyalgia, grief, heart disease, heart failure, hepatitis A, hepatitis B, hepatitis C, HIV/AIDS, hyperthyroidism (overactive thyroid), hypothyroidism (underactive thyroid), inflammatory bowel disease (IBD), liver disease, low vitamin D, lupus, mononucleosis, multiple sclerosis, obesity, Parkinson's disease, physical abuse, emotional abuse, polymyalgia rheumatica, pregnancy, rheumatoid arthritis, sleep apnea, stress, and traumatic brain injury. Such conditions also hinder a subject's ability to perform physical exercise and work and thus hinder a subject's abilities to improve health, increase endurance, increase muscle mass, reduce fat, improve muscle to fat ratio, improve athletic conditioning, feel healthier, feel happier, and get more physically fit through physical exercise.

The symptoms and etiology of percarbonate-improvable conditions that can be ameliorated by the administration of percarbonate. The formulations described herein are described in the “Handbook of Diseases 3rd Edition by Springhouse Corporation”, which is fully incorporated by reference herein.

Thus, methods of using percarbonate are also described herein. The methods generally comprise orally administering to a subject an effective amount of percarbonate to achieve the desired effect. The effective amount can be administered before, during or after physical exercise or work. In the case of treating diseases and conditions, the effective amount may be administered once per day, multiple times per day, or continuously through the day via time release, delayed release, or extended release formulations.

The dosage for oral use of the percarbonate ranges from 1 mg to 30,000 mg per day, which can be split into one or more dosages. For exercise performance enhancing purposes, the formulation is administered (for example, by ingestion) prior to exercise, for example any time between 3 days prior to exercise and right before exercise. In certain implementations, the formulation is administered one hour before exercise. In other implementations, the formulation is administered during exercise, or any time after exercise to assist with muscle building, lactic acid removal, and recovery. For purposes of treating, preventing or curing a percarbonate-improvable condition, the formulation is administered in a pharmaceutically effective amount to treat as needed said condition or disease.

Compositions and/or formulations of the present invention may be administered in any form, including one of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof for example. Compositions and/or formulations of the present invention may also include an acceptable additive (e.g. one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or an acceptable carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof).

Implementations of percarbonates and percarbonate compositions may conveniently be presented in unit dose form. Unit dose formulations may be those containing a daily dose or unit, a daily sub-dose, or an appropriate fraction thereof, of the administered components as described herein. Thus, a unit dose formulation of the percarbonates and percarbonate compositions comprises 1 mg to 30,000 mg of the percarbonate, 1 mg to 15,000 mg of the percarbonate, 1 mg to 7,500 mg of the percarbonate, 1 mg to 5,000 mg of the percarbonate, 1,000 mg to 30,000 mg of the percarbonate, 1,000 mg to 15,000 mg of the percarbonate, 1,000 mg to 7,500 mg of the percarbonate, 1,000 mg to 5,000 mg of the percarbonate, 1,500 mg to 30,000 mg of the percarbonate, 1,500 mg to 15,000 mg of the percarbonate, 1,500 mg to 7,500 mg of the percarbonate, 1,500 mg to 5,000 mg of the percarbonate, 1,500 mg to 3,000 mg of the percarbonate, 1,500 mg to 2,500 mg of the percarbonate, or about 2,000 mg of the percarbonate.

In some aspects, a unit dose formulation comprises percarbonate and a dissolution enhancer such as sodium carbonate, potassium carbonate, magnesium carbonate, calcium carbonate, zinc carbonate, copper carbonate and iron carbonate, sodium bicarbonate, potassium bicarbonate, magnesium bicarbonate, calcium bicarbonate, zinc bicarbonate, copper bicarbonate and iron bicarbonate. In some embodiments, the unit dose formulation comprises the percarbonate admixed with at least pharmaceutically acceptable additive(s).

The unit dose formulation may be in a form suitable for administration by standard routes. In general, the unit dose formulation may be administered, by non-limiting example, by the topical (including buccal and sublingual), transdermal, oral, rectal, ophthalmic (including intravitreal or intracameral), nasal, vaginal, and/or parenteral (including subcutaneous, intramuscular, intravenous, intradermal, intratracheal, and epidural) routes. In some implementations, the unit dose formulation comprises the percarbonate in a coated, microencapsulated, or other form that prevent dissolution of percarbonate in water or any other solvent in which the percarbonate is administered. In another embodiment, the unit dose formulation comprises the percarbonate dissolved or in suspension in a suitable liquid, such as, but not limited to, glycerol, water, various oils, and alcohol. Examples include coating percarbonate particles by lecithin, carnauba wax, shellac (for enteric release) or a fatty acid such as stearic acid. A unit dose formulation may also include the percarbonate in a time release, delayed release or prolonged release form.

For the exemplary purposes of this disclosure, oral delivery may be a particularly advantageous delivery route for administration of the percarbonates and percarbonate compositions to humans and animals. In such implementations, the percarbonate composition is a pharmaceutical composition optionally formulated with appropriate pharmaceutically acceptable additives to facilitate oral administration of the composition.

In some implementations, the percarbonate composition further comprises additional supplements or ingredients that enhance the effectiveness of percarbonate. These supplements or ingredients include, but are not limited to: 1,3-DMAA, 1,3-DMBA, 1,4-Butanediol, 1,4-DMAA, 1-Androsterone, 1-Epiandrosterone, 19-nor-DHEA,4-Androsterone, 5-deca zol, 5-HTP, 5aOHP, 6-Bromo, 7,8-benzoflavone, 7-alphahydroxy-DHEA, 7-beta-hydroxy-DHEA, 7-Keto-DHEA, 7-methoxyflavone, abscess root, abuta, acacia rigidula, acai, acerola, ackee, aconite, activated charcoal, active hexose correlated compound (AHCC), adenosine, adrafinil, adrenal extract, adrue, aegeline, african wild potato, agar, agaricus mushroom, agave, agmatine, agrimony, Ajuga nipponensis, alanine, Albizia julibrissin, alchemilla, alder buckthorn, aletris, alfalfa, algal oil, algin, alkanna, allspice, aloe, alpha-GPC, alpha-ketoglutarate (AKG), alpha-linolenic acid (ALA), alpha-lipoic acid, alpine ragwort, alpinia, amaranth, ambrette, American adder's tongue, American chestnut, American dogwood, American elder, American ginseng, American hellebore, American ivy, American mistletoe, American pawpaw, American spikenard, American white water lily, andarine, andiroba, andrographis, androstenediol, androstenedione, androstenetrione, androsterone, angel's trumpet, Angelica archangelica, angostura, anhydrous crystalline maltose, anise, annatto, antineoplastons, antioxidants, apoaequorin, apple, apple cider vinegar, apple polyphenols, apricot, apricot kernel, arabinoxylan, arenaria rubra, arimistane, aristolochia, arnica, arrach, arrowroot, arsenic, artemisia herba-alba, artichoke, arum, asafoetida, asarabacca, Ascophyllum nodosum, ascorbigen, ash, ashitaba, ashwagandha, asian water plantain, asparagus, asparagus racemosus, aspartic acid, aspen, astaxanthin, astragalus, atlantic cedar, atractylodes, autumn crocus, avens, avocado, avocado soy unsaponifiables (ASU), avocado sugar extract, ayahuasca, babassu, bach flower remedies, Bacillus coagulans, bael, baikal skullcap, ba ji tian, bamboo, banaba, banana, baobab, barley, basil, bay leaf, bayberry, bear's garlic, bee pollen, bee venom, beer, beeswax, beet, belladonna, benfotiamine, benzoin, berberine, bergamot, beta-alanine, beta-carotene, betacryptoxanthin, beta-glucans, beta-hydroxybutyrate (BHB), beta-methylphenethylamine (BMPEA), beta-sitosterol, betaine anhydrous, betaine hydrochloride, betel nut, beth root, betony, Bifidobacterium animalis subsp, lactis, Bifidobacterium bifidum, Bifidobacterium breve, Bifidobacterium longum, bilberry, biotin, birch, bishop's weed, bismuth, bismuth nitrate, bistort, bitter almond, bitter melon, bitter milkwort, bitter orange, bitter yam, bittersweet nightshade, black alder, black bryony, black cohosh, black currant, black haw, black hellebore, black hoof mushroom, black horehound, black mulberry, black mustard, black nightshade, black pepper, black psyllium, black raspberry, black rice, black root, black seed, black tea, black walnut, blackberry, blackthorn, blessed thistle, blond psyllium, bloodroot, blue cohosh, blue flag, blue-green algae, blueberry, bog bilberry, bog labrador tea, bogbean, bois de rose oil, boldo, boneset, borage, boron, boswellia serrata, bovine cartilage, bovine colostrum, boxwood, branched-chain amino acids (BCAA), breadfruit, brewer's yeast, brickellia, bridelia, broccoli, broccoli sprout, bromelain, brown rice, brussels sprout, bryonia, buchu, buck's-horn plantain, buckhorn plantain, buckwheat, bugle, bugleweed, Bulbine natalensis, bulbous buttercup, bupleurum, burdock, burning bush, burr marigold, butcher's broom, butea superba, butterbur, buttercup, butternut, butylated hydroxytoluene (BHT), cabbage, cade, caffeic acid, caffeine, cajeput oil, calabar bean, calabash chalk, calamint, calamus, calanus oil, calcium, calcium D-glucarate, Calea zacatechichi, calendula, california poppy, calotropis, calumba, camphor, camu camu, canada balsam, canadian fleabane, canadian hemp, canaigre, cananga oil, cannabichromene (CBC), cannabidiol (CBD), cannabidivarin (CBDV), cannabigerol (CBG), cannabinol (CBN), cannabis, canola oil, canthaxanthin, capers, caprylic acid, capsicum, caralluma, caraway, carbon 60 (C60), cardamom, cardarine, carlina, carnosine, carob, carqueja, carrageenan, carrot, cascara sagrada, cascarilla, casein peptides, casein protein, cashew, cassava, cassia auriculata, cassia cinnamon, cassia nomame, cassie absolute, castor bean, castoreum, cat's claw, cat's foot, catechu, catnip, catuaba, cauliflower, celery centaury, cereus, cesium, cetylated fatty acids (CFAs), ceylon cinnamon, ceylon leadwort, cha de bugre, chaga, chanca piedra, chaparral, chaulmoogra, cheken, chelation therapy products, chenopodium oil, chervil, chia, chickweed, chicory, Chinese cucumber, Chinese mallow, Chinese prickly ash, chirata, chitosan, chive, chlorella, chlorine dioxide, chlorophyll, chlorophyllin, chokeberry, chondroitin sulfate, chromium, chrysanthemum, chrysin, chuchuhuasi, chymotrypsin, cilantro, cinchona, Cissus quadrangularis, Cistanche deserticola, citicoline, citric acid, citronella oil, clary sage, clay, Clematis recta, clivers, clove, clown's mustard plant, clubmoss, cnidium, cobalt, coca, cocillana, cocoa, coconut, coconut oil, coconut water, cod liver oil, codonopsis, coffee, coffee charcoal, cola nut, coleus, collagen peptides, collagen type I (native), collagen type II (native), collard, colloidal minerals, colloidal silver, colocynth, coltsfoot, columbine, Combretum micranthum, comfrey, common stonecrop, condurango, conjugated linoleic acid (CLA), copaiba balsam, copper, coral, cordyceps, coriander, corkwood tree, corn poppy, corn silk, cornflower, Corydalis yanhusuo, costus, cotton, couch grass, cowhage, cowslip, cramp bark, cranberry, creatine, croton seeds, cubebs, cucumber, cudweed, cumin, cursed buttercup, cyanostane, cyclamen, cypress, d-mannose, daffodil, damiana, dandelion, danshen, date palm, Datura wrightii, deanol, deer velvet, delta-8-tetrahydrocannabinol (delta-8-THC), delta-9-tetrahydrocannabinol (delta-9-THC), dendrobium, desert parsley, devil's claw, devil's club, dhea, diacylglycerol, diatomaceous earth, diindolylmethane, diiodothyronine, dill, dimethylglycine (DMG), dimethylhexylamine (DMHA), dimethylsulfoxide (DMSO), diosmin, divi-divi, docosahexaenoic acid (DHA), dodder, dolomite, dong quai, douglas fir, dragon fruit, dragon's blood, duckweed, dulse, durabolin, durian, dusty miller, dwarf elder, dwarf pine needle, dyer's broom, dymethazine, eastern hemlock, eastern red cedar, ecdysteroids, echinacea, Ecklonia cava, eicosapentaenoic acid (EPA), elderberry, elderflower, elecampane, elemi, eleuthero, ellagic acid, elm bark, emu oil, English adder's tongue, English horsemint, English ivy, English walnut, ephedra, epiandrosterone, epistane, equol, ergot, ergothioneine, eryngo, eucalyptus, Euphorbia cyparissias, Euphorbia hirta, European barberry, European buckthorn, European chestnut, European five-finger grass, European mandrake, European mistletoe, Eurycoma longifolia, evening primrose, evodia, eyebright, Fadogia agrestis, false unicorn, fennel, fenugreek, fermented milk, fermented wheat germ extract, fever bark, feverfew, ficin, field scabious, fig, figwort, fir, fireweed, fish oil, flaxseed, flaxseed oil, fluoride, fly agaric mushroom, fo-ti, folic acid, fool's parsley, forget-me-not, forsythia, foxglove, frankincense, fringetree, frostwort, fructo-oligosaccharides (FOS), Fucus vesiculosus, fulvic acid, fumitory, galacto-oligosaccharides (GOS), galbanum, Galphimia glauca, gamboge, gamma butyrolactone (GBL), gamma-aminobutyric acid (GABA), gamma-hydroxybutyrate (GHB), gamma-linolenic acid (GLA), gamma-oryzanol, garcinia, garden cress, gardenia, garlic, gelatin, gelsemium, genistein combined polysaccharide, gentian, German chamomile, German ipecac, German sarsaparilla, germander, germanium, ginger, ginkgo, globe flower, globemallow, glossy privet, glucomannan, glucosamine, glucuronolactone, glutaurine, glutathione, glycerol, glycine, glycolic acid, glycomacropeptide, glyconutrients, goa powder, goat's rue, goji, golden ragwort, goldenrod, goldenseal, goldthread, gossypol, gotu kola, goutweed, grains of paradise, grape, grapefruit, gravel root, graviola, great plantain, greater burnet, greater celandine, greek sage, green coffee, green tea, Griffonia simplicifolia, ground ivy, ground pine, groundsel, guaiac wood, guar gum, guarana, guarumo, guava, guggul, gum arabic, gumweed, gymnema, halodrol-50, haronga, hartstongue, Hawaiian baby woodrose, hawthorn, hazelnut, heart's ease, heather, hedge mustard, hedge-hyssop, hemlock, hemlock water dropwort, hemp, hemp agrimony, hempnettle, henbane, henna, herb Paris, herb Robert, Hercules club, hesperidin, hexadrone, hexylamine, hibiscus sabdariffa, higenamine, histidine, holly, hollyhock, holy basil, homotaurine, honey, honeysuckle, hoodia, hops, hordenine, horny goat weed, horse chestnut, horsemint, horseradish, horsetail, hound's tongue, houseleek, hu zhang, humic acid, huperzine A, hyacinth bean, hyaluronic acid, hydrangea, hydrazine sulfate, hydroxymethylbutyrate (HMB), hyperimmune egg, hyssop, iboga, Iceland moss, idebenone, ignatius bean, immortelle, Indian cassia, Indian gooseberry, Indian long pepper, Indian snakeroot, indigo pulchra, indium, indole-3-carbinol, inosine, inositol, inositol nicotinate, inulin, iodine, ip-6, ipecac, iporuru, ipriflavone, iron, Irvingia gabonensis, isatis, isopropylnorsynephrine, ivy gourd, jaborandi, jackfruit, jalap, Jamaican dogwood, jambolan, Japanese apricot, Japanese mint, Japanese persimmon, jasmine, java tea, Javanese turmeric, jequirity, jewelweed, jiaogulan, jimson weed, job's tears, jojoba, juniper, Justicia pectoralis, K2/spice, kale, kamala, kaolin, karaya gum, kava, kefir, ketogenic diet, khat, khella, kinetin, kiwi, knotweed, kohlrabi, kombucha, Korean pine, kousso, kratom, krill oil, kudzu, L-arginine, L-citrulline, L-cysteine, L-ornithine-L-aspartate, labdanum, laburnum, lactase, lactic acid, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus crispatus, Lactobacillus delbrueckii, Lactobacillus gasseri, Lactobacillus helveticus, Lactobacillus jensenii, Lactobacillus johnsonii, lactoferrin, lady's bedstraw, laminaria, larch arabinogalactan, larch turpentine, lathyrus, Latilactobacillus sakei, laurelwood, lauric acid, lavender, lavender cotton, laxogenin, lemon, lemon balm, lemon eucalyptus, lemon verbena, lemongrass, lentinan, lesser celandine, levant berry, Levilactobacillus brevis, licorice, Ligandrol, Ligilactobacillus salivarius, lily-of-the-valley, lime, limonene, Limosilactobacillus fermentum, Limosilactobacillus reuteri, linden, lingonberry, lion's mane mushroom, lipase, lithium, liver extract, liverwort, lobelia, logwood, Lorenzo's oil, lotus, lousewort, lovage, luffa, lunasin, lungmoss, lungwort, lupin, lutein, lychee, lycopene, lysine, M1-4ADD, maca, macadamia nut, mace, Madagascar periwinkle, madder, magnesium, magnolia, maidenhair fern, maitake mushroom, malabar nut, male fern, malic acid, mallow, manaca, Manchurian thorn, manganese, mangosteen, manna, maqui, maral root, maritime pine, marjoram, marsh blazing star, marsh labrador tea, marsh marigold, marshmallow, masterwort, mastic, meadowsweet, medium chain triglycerides (MCTs), melanotan, melatonin, mentabolan, mercury herb, mesoglycan, methasterone, methionine, methoxydienone, methoxylated flavones, methyldiazinol, methylstenbolone, methylsulfonylmethane (MSM), methylsynephrine, mezereon, milk thistle, miracle fruit, molybdenum, moneywort, monolaurin, Monterey pine, moringa, Mormon tea, motherwort, mountain ash, mountain flax, mountain laurel, mouse-ear hawkweed, mugwort, muira puama, mullein, musk, myrcia, myrrh, myrtle, N, N-DMPEA, N-acetyl cysteine (NAC), N-methyltyramine, nasturtium, nattokinase, neem, nerve root, new jersey tea, New Zealand green-lipped mussel, niacinamide, niauli oil, nickel, nicotinamide riboside, nikko maple, noni, northern prickly ash, Norway spruce, nutmeg, nux vomica, oak moss, oats, octacosanol, octopamine, oleander, oleic acid, olive, olive oil, omega-6 fatty acids, onion, oolong tea, orchic extract, oregano, Oregon grape, oriental arborvitae, ornamental marigold, ornithine, ornithine ketoglutarate (OKG), orris, oscillococcinum, osha, ostarine, ostrich fern, oswego tea, ox-eye daisy, padang cassia, pagoda tree, palm oil, palmitoylethanolamide (PEA), Panax ginseng, Panax notoginseng, pancreatic enzyme products, pangamic acid, pantethine, pao pereira, papain, papaya, para-aminobenzoic acid (PABA), pareira, parsley, parsley piert, parsnip, partridgeberry, passion flower, pata de vaca, patchouli oil, Pau D'Arco, pea protein, peanut oil, pear, pectin, pedunculate oak, pellitory, pellitory-of-the-wall, pennyroyal, peony, peppermint, perilla, perillyl alcohol, periwinkle, peru balsam, peyote, Phaseolus vulgaris, pheasant's eye, phellodendron, phenethylamine (PEA), phenibut, phenpromethamine, phlorizin, phosphate salts, phosphatidylcholine, phosphatidylserine, phytase, picamilon, picrorhiza, pimpinella, Pinellia ternata, pink root, pipsissewa, piracetam, pitcher plant, plant sterols, pleurisy root, plum, podophyllum, poinsettia, poison ivy, pokeweed, polarity therapy, policosanol, polydextrose, Polypodium leucotomos, pomegranate, poplar, poppy seed, poria mushroom, potassium, potato, potentilla, pregnenolone, premorse, prickly pear cactus, procaine, progesterone, proline, propolis, proteolytic enzymes (proteases), psilocybin, pu-erh tea, pulsatilla, pumpkin, purple loosestrife, purple nut sedge, pygeum, pyrethrum, pyruvate, quassia, quebracho blanco, queen's delight, quercetin, quillaia, quince, quinoa, Rabdosia rubescens, radish, raspberry ketone, Rauvolfia vomitoria, rauwolscine, red clover, red maple, red raspberry, red sandalwood, red soapwort, red yeast rice, red-spur valerian, reed herb, rehmannia, reishi mushroom, resveratrol, rhatany, rhodiola, rhubarb, riboflavin, ribose, rice bran, rice bran arabinoxylan compound, rice (protein, RNA, or DNA), rock rose, roman chamomile, rooibos, rose geranium oil, rose hip, rosemary, royal jelly, rue, rupturewort, rusty-leaved rhododendron, rutin, rye grass, S-23, Saccharomyces boulardii, safed musli, safflower, saffron, sage, saigon cinnamon, salacia, salatrim, salep, Salvia divinorum, samphire, sandy everlasting, sangre de grado, sanicle, sarsaparilla, sassafras, savin tops, saw palmetto, scarlet pimpernel, sceletium, schisandra, schizonepeta, scopolia, scotch broom, scotch thistle, scurvy grass, sea buckthorn, sea moss, secretin, Securinega suffruticosa, selenium, self-heal, senega, senna, serine, serrapeptase, sesame, sessile oak, shark cartilage, shark liver oil, shea butter, shellac, shepherd's purse, shiitake mushroom, Siberian cocklebur, Sida cordifolia, silicon, simaruba, sitostanol, skullcap, skunk cabbage, slippery elm, smartweed, smooth alder, snake skin, sodium, sodium bicarbonate, sodium tetrachloroaurate, Solomon's seal, sorghum, sorrel, soy, soybean oil, Spanish broom, Spanish origanum oil, spearmint, spinach, spiny restharrow, spleen extract, spotted geranium, squalamine, squill, St. John's wort, star anise, star of bethlehem, stavesacre, stenabolic, Stereospermum, stevia, stinging nettle, stone root, storax, strawberry, Streptococcus thermophilus, strontium, strophanthus, succinate, sulbutiamine, sulforaphane, sulfur, suma, sumbul, summer savory, sundew, sunflower oil, superoxide Dismutase (SOD), swallowroot, swamp milkweed, sweet almond, sweet Annie, sweet cherry, sweet cicely, sweet clover, sweet gale, sweet orange, sweet sumac, sweet vernal grass, sweet violet, sweet woodruff, Syrian rue, tamarind, Tamarix dioica, tangerine, tannic acid, tansy, tansy ragwort, tapioca, tarragon, tart cherry, tartaric acid, tea tree oil, teazle, terminalia, testolone, tetrahydrocannabivarin (THCV), theacrine, theaflavin, threonine, thuja, thunder god vine, thyme, thymus extract, thyroid extract, tianeptine, timothy grass, tin, Tinospora cordifolia, tiratricol, tocotrienols, tolu balsam, tomato, tonka bean, toothed clubmoss, tormentil, tragacanth, trailing arbutus, transfer factor, traveler's joy, tree of heaven, tree tobacco, tree turmeric, trendione, tribulus, Trichopus zeylanicus, tronadora, trypsin, tung seed, turkey corn, turkey tail mushroom, turmeric, turpentine oil, turtlehead, tylophora, tyramine, umckaloabo, usnea, uva ursi, uzara, valerian, vanadium, vanilla, verbena, veronica, vetiver, Vietnamese coriander, vinpocetine, vitamin A, vitamin B1, vitamin B2, vitamin B6 (or a salt, ester, or amide suitable for human ingestion thereof), vitamin B8 (or a salt, ester, or amide suitable for human ingestion thereof), vitamin B9 (or a salt, ester, or amide suitable for human ingestion thereof), vitamin B12 (or a salt, ester, or amide suitable for human ingestion thereof), vitamin C, vitamin D, vitamin E, vitamin K, vitamin O, wafer ash, wahoo, wallflower, wasabi, water avens, water dock, water hemlock, watercress, wheat bran, wheatgrass, whey protein, white dead nettle flower, white hellebore, white horehound, white lily, white mulberry, white mustard, white oak, white pepper, white sandalwood, wild carrot, wild cherry, wild daisy, wild indigo, wild lettuce, wild mint, wild thyme, wild yam, willard water, willow bark, wine, winter cherry, winter savory, wintergreen, witch hazel, wood anemone, wood sage, wood sorrel, wormseed, wormwood, xanthan gum, xanthoparmelia, xylitol, yarrow, yellow dock, yellow loosestrife, yellow toadflax, yerba mansa, yerba mate, yerba santa, yew, yin chen, ylang ylang oil, yogurt, yohimbe, yucca, zeaxanthin, zedoary, zinc, zizyphus, L-carnitine (or a salt, ester, or amide suitable for human ingestion thereof), acetyl-L-carnitine (or a salt, ester, or amide suitable for human ingestion thereof), propionyl-L-carnitine (or a salt, ester, or amide suitable for human ingestion thereof), aniracetam, piracetam, phenylpiracetam, Bacopa monnieri, cannabidiol (CBD), Celastris paniculatus, centrophenoxine, Clitoria ternatea, coluracetam, Convolvulus pluricaulis, ubiquinone (for example, Coenzyme Q10), creatine (or a salt, ester, or amide suitable for human ingestion thereof), choline (or a salt, ester, or amide suitable for human ingestion thereof), dimethylethanolamine (DMAE), forskolin, Ginkgo biloba, guarana, kanna, kava kava, L-glutamine (or a salt, ester, or amide suitable for human ingestion thereof), L-phenylalanine (or a salt, ester, or amide suitable for human ingestion thereof), L-theanine (or a salt, ester, or amide suitable for human ingestion thereof), L-tryptophan (or a salt, ester, or amide suitable for human ingestion thereof), lecithin (or a salt, ester, or amide suitable for human ingestion thereof), lemon balm, lion's mane mushroom, magnolia, medium chain triglycerides, reduced nicotinamide adenine dinucleotide (NADH), nefiracetam, nicotine, arecoline, noopept, oatstraw, oxiracetam, passion flower, phenibut, phosphatidylcholine, phosphatidylserine, picamilon, pine bark extract, pramiracetam, pyrroloquinoline quinone (PQQ), pterostilbene, resveratrol, rosemary, Rhodiola rosea, S-adenosyl methionine (SAMe), schizandrol-A, St. John's wort, sulbutiamine, taurine (or a salt, ester, or amide suitable for human ingestion thereof), L-tyrosine (or a salt, ester, or amide suitable for human ingestion thereof), N-acetyl-L-tyrosine (or a salt, ester, or amide suitable for human ingestion thereof), uridine, valerian, vinpocetine, thiamine (or a salt, ester, or amide a salt, ester, or amide suitable for human ingestion thereof), niacin (or a salt, ester, or amide suitable for human ingestion thereof), pantothenic acid (or a salt, ester, or amide suitable for human ingestion thereof), and yerba mate.

It should be appreciated that any of the components of particular implementations of percarbonate (sodium percarbonate or other percarbonates) and percarbonate compositions may be used as supplied commercially, or may be preprocessed by, by non-limiting example, any of the methods and techniques of agglomeration, air suspension chilling, air suspension drying, balling, coacervation, comminution, compression, pelletization, cryopelletization, extrusion, granulation, homogenization, inclusion compoundation, lyophilization, melting, mixed, molding, pan coating, solvent dehydration, sonication, spheronization, spray chilling, spray congealing, spray drying, or other processes known in the art depending in part on the dosage form desired. The various components may also be pre-coated or encapsulated as known in the art. It will also be clear to one of ordinary skill in the art that appropriate additives may also be introduced to the composition or during the processes to facilitate the preparation of the dosage forms, depending on the need of the individual process.

Those of ordinary skill in the art will be able to readily select manufacturing equipment and pharmaceutically acceptable additives or inert ingredients to manufacture implementations of percarbonate and percarbonate compositions. For the exemplary purposes of this disclosure, some examples of pharmaceutically acceptable additives or inert ingredients and manufacturing processes are included herein, particularly those that relate to manufacture of implementations of percarbonate and percarbonate compositions in tablet form or powder form.

Notwithstanding the specific examples given, it will be understood that those of ordinary skill in the art will readily appreciate how to manufacture implementations of percarbonate and percarbonate compositions according to the other methods of administration and delivery disclosed in this document.

Particular implementations of percarbonate and percarbonate compositions include a lubricant. Lubricants are any anti-sticking agents, glidants, flow promoters, and the like materials that perform a number of functions in tablet manufacture, for example, such as improving the rate of flow of the tablet granulation, preventing adhesion of the tablet material to the surface of the dies and punches, reducing interparticle friction, and facilitating the ejection of the tablets from the die cavity. Lubricants may comprise, for example, magnesium stearate, calcium stearate, talc, and colloidal silica.

Particular implementations of percarbonate and percarbonate compositions include a binder. Binders are any agents used to impart cohesive qualities to powdered material through particle-particle bonding. Binders may include, for example, matrix binders (e.g., dry starch, dry sugars), film binders (e.g., celluloses, bentonite, sucrose), and chemical binders (e.g., polymeric cellulose derivatives, such as methyl cellulose, carboxy methyl cellulose, and hydroxy propyl cellulose); and other sugar, gelatin, non-cellulosic binders and the like.

Disintegrators may be used in particular implementations of percarbonate and percarbonate compositions to facilitate the breakup or disintegration of tablets after administration. Disintegrators may include, for example, starch, starch derivatives, clays (e.g., bentonite), algins, gums (e.g., guar gum), cellulose, cellulose derivatives (e.g., methyl cellulose, carboxymethyl cellulose), croscarmellose sodium, croscarmellose cellulose, and other organic and inorganic materials.

Implementations of percarbonate and percarbonate compositions may also include diluents, or any inert substances added to increase the bulk of a percarbonate to make a tablet a practical size for compression. Diluents may include, for example, calcium phosphate, calcium sulfate, lactose, mannitol, magnesium stearate, potassium chloride, and citric acid, among other organic and inorganic materials.

Buffering agents may be included in implementations of percarbonate and percarbonate compositions as well and may be any one of an acid and a base, where the acid is, for example, propionic acid, p-toluenesulfonic acid, salicylic acid, stearic acid, succinic acid, tannic acid, tartaric acid, thioglycolic acid, or toluenesulfonic acid, and the base is, for example, ammonium hydroxide, potassium hydroxide, sodium carbonate, potassium carbonate, sodium hydroxide, sodium hydrogen carbonate, aluminum hydroxide, calcium carbonate, and other organic and inorganic chemicals.

Percarbonates and percarbonate compositions may also be administered through use of amphipathic lipid delivery systems (such as liposomes and unilamellar vesicles), caplet systems, oral liquid systems, parenteral and/or intravenous systems, topical systems (creams, gels, transdermal patches, etc.), intranasal systems, rectal or vaginal systems, and many other delivery methods and/or systems known to those of ordinary skill in the art. Those of ordinary skill in the art will readily be able to select additional pharmaceutically acceptable additives to enable delivery of implementations of a pharmaceutical composition from the disclosure in this document.

With respect to delivery of particular implementations and uses of percarbonates and percarbonate compositions, for the exemplary purposes of this disclosure, tablets may be utilized. Tablets are any solid pharmaceutical dosage form containing a pharmaceutically acceptable active agent or agents to be administered with or without suitable pharmaceutically acceptable additives and prepared either by compression or molding methods well known in the art. Tablets have been in widespread use and remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be, for example, round, oval, oblong, cylindrical, rectangular, or triangular. The tablets may be optionally scored so that they may be separated into different dosages. They may differ greatly in size and weight depending on the amount of the pharmaceutically acceptable active agent or agents present and the intended route of administration. They are divided into two general classes: compressed tablets and molded tablets.

Tablets and other orally discrete dosage forms, such as capsules, cachets, pills, granules, pellets, beads, and particles, for example, may optionally be coated with one or more enteric coatings, seal coatings, film coatings, barrier coatings, compress coatings, fast disintegrating coatings, or enzyme degradable coatings for example. Multiple coatings may be applied for desired performance. Further, dosage forms may be designed for, by non-limiting example, immediate release, pulsatile release, controlled release, extended release, delayed release, targeted release, synchronized release, or targeted delayed release. For release/absorption control, carriers may be made of various component types and levels or thicknesses of coats. Such diverse carriers may be blended in a dosage form to achieve a desired performance. In addition, the dosage form release profile may be affected by a polymeric matrix composition, a coated matrix composition, a multiparticulate composition, a coated multi-particulate composition, an ion-exchange resin-based composition, an osmosis-based composition, or a biodegradable polymeric composition.

The contents of all linked references, references, patents, and published patent applications cited throughout this specification, if any, are incorporated herein by reference in their entirety for all purposes.

### EXAMPLES

The present disclosure is further illustrated by the following examples that should not be construed as limiting.

### Example 1

The following four subjects were directed to ingest 2 grams of sodium percarbonate in the form of two 000 capsules (each capsule comprising 1,000 mg sodium percarbonate) immediately before a session of exercise: a 48-year-old male (former fighter); a 28-year-old male (professional mixed martial arts (MMA) fighter); a 26-year-old male (professional MMA fighter); a 29-year-old male (diabetic; trains regularly at the gym); and a 34-year-old female.

All subjects described increased endurance, strength, reduced lactic acid burn, and improved recovery during and after the session of exercise.

In particular, the 29-year-old male subject with diabetes described his experience of his exercise routine after ingesting 2 grams of sodium percarbonate as follows: “First impressions on stamina/endurance ingredient. It works, like as simple as that, does exactly what you said it was. No increase in energy or focus anything like that but my breathing feels good, recovery between sets is quicker and I've made it through my entire back workout and cardio and still feel like I could keep going (which on this cut I'm normally dead half way through my sets)”.

The 34-year-old female subject ingested 2 grams of sodium percarbonate before a cardio session on the StairMaster® but after she performed leg training. She described that she definitely “felt” like her legs didn't ache as much as usual, and her endurance levels were definitely better than normal for exercising without breaking fast.

### Example 2

A 59-year-old male who regularly kayaks ingested 2 grams of sodium percarbonate in the form of two 000 capsules (each capsule comprising 1,000 mg sodium percarbonate) just before starting a routine kayaking trip. He noted greatly increased endurance and did not feel any burning in his muscles after going through the same routine.

1. A composition for oral administration comprising a percarbonate.

2. The composition of claim 1, wherein the percarbonate is sodium percarbonate.

3. The composition of claim 1 further comprising a dietary ingredient, a food additive, or a pharmaceutically acceptable additive.

4. The composition of claim 3, wherein the pharmaceutically acceptable additive comprises one or more of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and a carrier.

5. The composition of claim 4, wherein the pharmaceutically acceptable additive comprises one or more of calcium phosphate, cellulose, stearic acid, croscarmellose cellulose, magnesium stearate, and silicon dioxide.

6. The composition of claim 1, wherein the composition is in capsule form.

7. A dietary supplement comprising a therapeutically effective dose of a percarbonate.

8. The dietary supplement of claim 7, wherein the percarbonate is sodium percarbonate.

9. The dietary supplement of claim 8, wherein the amount of percarbonate in a dosage is about 1 mg to about 30,000 mg.

10. The dietary supplement of claim 7, wherein the dietary supplement is in the form of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, or combinations thereof.

11. The dietary supplement of claim 7, additionally comprising one or more of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and an acceptable carrier.

12. The dietary supplement of claim 11, wherein the acceptable carrier comprises an excipient, a lubricant, a binder, a disintegrator, a diluent, an extender, a solvent, a suspending agent, a dissolution aid, an isotonic agent, a buffering agent, a soothing agent, an amphipathic lipid delivery system, or combinations thereof.

13. A pharmaceutical composition comprising a therapeutically effective dose of percarbonate.

14. The pharmaceutical composition of claim 13, where in the daily dosage of percarbonate is about 1 mg to about 30,000 mg in one or multiple doses.

15. The pharmaceutical composition of claim 13, wherein the percarbonate is sodium percarbonate.

16. The pharmaceutical composition of claim 13, wherein the dose is in the form of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof.

17. The pharmaceutical composition of claim 13, wherein the dose additionally comprises one or more of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and an acceptable carrier.

18. The pharmaceutical composition of claim 17, wherein the acceptable carrier comprises an excipient, a lubricant, a binder, a disintegrator, a diluent, an extender, a solvent, a suspending agent, a dissolution aid, an isotonic agent, a buffering agent, a soothing agent, an amphipathic lipid delivery system, or combinations thereof.

19. A method of administering a percarbonate to a subject wherein an effective amount of the percarbonate is administered to the subject.

20. The method of claim 19, wherein a daily dosage of percarbonate of about 1 mg to about 30,000 mg in one or multiple doses is administered to the subject.

21. The method of claim 20, wherein the daily dosage is administered in one dose.

22. The method of claim 20, wherein the daily dosage is administered in two to four doses.

23. The method of claim 19, wherein the percarbonate is sodium percarbonate.

24. The method of claim 20, wherein the dose is in the form of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof.

25. The method of claim 20, wherein the dose additionally comprises one or more of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and an acceptable carrier.

26. The method of claim 25, wherein the acceptable carrier comprises an excipient, a lubricant, a binder, a disintegrator, a diluent, an extender, a solvent, a suspending agent, a dissolution aid, an isotonic agent, a buffering agent, a soothing agent, an amphipathic lipid delivery system, or combinations thereof.

27. The method of claim 19, wherein the percarbonate is orally administered to the subject.

28. The method of claim 19, wherein the percarbonate is orally administered to the subject as a dietary supplement.

29. The method of claim 19, wherein the percarbonate is orally administered to the subject as a pharmaceutical composition.

30. The method of claim 19, wherein the percarbonate is administered for improving physical performance of the subject, increasing recovery from physical exertion of the subject, reducing lactic acid burn of the subject, and/or treating a percarbonate-improvable condition of the subject that causes fatigue and/or malaise.

31. The method of claim 30, wherein the percarbonate-improvable condition is one or more of: Alcohol use, Drug use, Malnutrition, Fatigue side effects from narcotics, antihistamines, chemotherapy, radiation therapy, pain medicines, heart medicines, and antidepressants, Lack of sleep, Lack of physical exercise, Overexhaustion, Adrenal insufficiency, Amyotrophic lateral sclerosis (ALS), Anemia, Anxiety disorders, Cancer, Myalgic encephalomyelitis/chronic fatigue syndrome (ME/CFS), Chronic infection or inflammation, Chronic kidney disease, COPD (chronic obstructive pulmonary disease), Emphysema, Coronavirus disease 2019 (COVID-19), Mood disorders, Depression (major depressive disorder), Diabetes, Fibromyalgia, Grief, Heart disease, Heart failure, Hepatitis A, Hepatitis B, Hepatitis C, HIV/AIDS, Hyperthyroidism (overactive thyroid), Hypothyroidism (underactive thyroid), Inflammatory bowel disease (IBD), Liver disease, Low vitamin D, Lupus, Mononucleosis, Multiple sclerosis, Obesity, Parkinson's disease, Physical abuse, Emotional abuse, Polymyalgia rheumatica, Pregnancy, Rheumatoid arthritis, Sleep apnea, Stress, and Traumatic brain injury.

32. The method of claim 19, wherein the percarbonate is administered to the subject before, during, and/or after physical work or exercise.
